(19) United States
(12) Patent Application Publication
(10) Pub. No.: US 2021/0283089 A1
(43) Pub. Date: Sep. 16, 2021

(54) Use of fumaric acid esters and pharmaceutically acceptable salts thereof in preparing pharmaceuticals for treating ferroptosis-related diseases

(71) Applicant: XIANGYA HOSPITAL CENTRAL SOUTH UNIVERSITY, Changsha (CN)

(72) Inventors: Yongheng CHEN, Changsha (CN); Ting LIU, Changsha (CN); Ye ZHANG, Changsha (CN); Shuang ZHAO, Changsha (CN); Zhuchu CHEN, Changsha (CN)

(21) Appl. No.: 17/335,202

(22) Filed: Jun. 1, 2021

Related U.S. Application Data
(63) Continuation-in-part of application No. PCT/CN2019/092233, filed on Jun. 21, 2019.

(30) Foreign Application Priority Data
Dec. 5, 2018 (CN) 2018114805625

(51) Int. Cl. A61K 31/225; A61P 1/16; A61K 9/00

(52) U.S. Cl. CPC A61K 31/225; A61K 9/0019; A61P 1/16

(57) ABSTRACT

Related is use of fumaric acid esters or pharmaceutically acceptable salts thereof in preparing a pharmaceutical for treating ferroptosis-related diseases. The fumaric acid esters includes dimethyl fumarate and/or monomethyl fumarate. The present application reveals, for the first time, the effect of fumaric acid ester and its pharmaceutically acceptable salt on a protein level of a ferroptosis key regulatory gene GPX4, provides a theoretical basis for the research of treatment strategies for the ferroptosis-related diseases, and provides a new outlet for preparing pharmaceuticals used for the ferroptosis-related diseases.

### TECHNICAL FIELD

The present application belongs to the field of chemical medicines, and in particular to use of fumaric acid esters and pharmaceutically acceptable salts thereof in preparing a pharmaceutical for treating ferroptosis-related diseases.

### BACKGROUND

Ferroptosis is a type of iron/active oxygen species-dependent cell death pathways, different from other death pathways such as apoptosis, necrosis and autophagy. The ferroptosis mainly manifests as cell lipid peroxidation and intracellular reactive oxygen species accumulation. Ferrostatin-1 and Erastin are recognized ferroptosis inhibitor and activator at present. It is indicated from researches that Erastin prevents a cystine from being ingested and reduced into a cysteine through inhibiting a cystine/glutamate transporter, and affects synthesis of glutathione. GSH is a necessary cofactor for a glutathione peroxidase to inhibit the formation of lipid reactive oxygen species. Herein glutathione peroxidase 4 (GPX4) is an important ferroptosis regulating factor, and may inhibit the occurrence of cell membrane lipid peroxidation. While the activity or expression of GPX4 is inhibited, the ferroptosis may be triggered. On the contrary, the increase of its activity or expression may inhibit the ferroptosis.

It is discovered from researches that a variety of diseases are related to the ferroptosis, including neurological diseases (Parkinson's disease, periventricular leukomalacia, Huntington's disease, Alzheimer's disease, motor neurodegeneration, stroke and traumatic brain injury, etc.; tumors (head and neck malignant tumors, breast cancer, esophageal cancer, liver cancer, pancreatic cancer, kidney cancer and diffuse large B-cell lymphoma, etc.); ischemia-reperfusion injury (myocardium, liver and kidney, etc.); acute renal failure and iron metabolism related diseases (atherosclerosis and diabetes, etc.); liver diseases (hemochromatosis, primary biliary cholangitis, liver fibrosis, etc.). Therefore, the ferroptosis is used as a target, and the ferroptosis may be targeted-inhibited by a pharmaceutical which may inhibit the ferroptosis, it has great significance to the treatment of ferroptosis related diseases.

Dimethyl fumarate (DMF, Tecfidera as its trade name) is a new-type pharmaceutical approved by the US Food and Drug Administration in March 2013 for treatment of multiple sclerosis. DMF and its main metabolic activity product in vivo monomethyl fumarate (MMF) may inhibit cellular reactive oxygen species (ROS) accumulation by activating a KEAP1-NRF2-ARE signal pathway which plays an important role in a cellular antioxidant mechanism, thereby withstanding the cell peroxidative damage.

CN107253927A discloses a type of a phospholipid hydrogen glutathione peroxidase (GPX4) activator and its use. It is verified by an in vitro enzyme activity test and a cell model experiment that this compound may be used as an activator of GPX4 to treat and prevent various inflammations, oxidative damage, neurodegenerative diseases and ferroptosis related diseases.

CN107890567A provides a new use of CDO1, which is that CDO1 is used to prepare a gastric cancer therapeutic pharmaceutical related to the ferroptosis. CDO1 is an important constituent part of c-Myb signal pathway in gastric cancer, and is an important metabolic node in the process of the ferroptosis. This disclosure reveals an action mechanism of CDO1 for regulating cysteine metabolism in Erastin-induced gastric cancer cell ferroptosis and a transcriptional regulation effect of c-Myb on CDO1 expression in the ferroptosis process for the first time. Overexpression of CDO1 may promote the ferroptosis process of gastric cancer cells. Therefore, the disclosure provides a theoretical basis for researching the treatment of gastric cancer based on the ferroptosis, and provides an embedded point for preparing a new gastric cancer therapeutic pharmaceutical.

CN108484527A discloses a new 10H-phenothiazine derivative that may inhibit ferroptosis. Through research in its structure optimization and structure-activity relationship, it is verified that the 10H-phenothiazine derivative has a good inhibiting effect on the ferroptosis and there exists a compound that shows a better therapeutic effect on a mouse focal cerebral ischemia model. The compound may be used as a main active ingredient for preparing a ferroptosis inhibitor. Both the compound and the inhibitor prepared by the compound have good medicinal potential; a preparation method for the new compound provided by the disclosure is simple, mild in reaction conditions, convenient for operation and control, low in energy consumption, high in yield, and low in cost, and may be suitable for industrial production. The prepared compound is higher in biological activity, strong in selectivity, and remarkable in drug-likeness, and has a broad market prospect.

The prior art seldom discloses strategies for treatment of the ferroptosis related diseases. Therefore, it is very meaningful to develop a new pharmaceutical that uses the ferroptosis as the target, has strong selectivity, and has significant efficacy for the treatment of the ferroptosis related diseases.

### SUMMARY

In view of problems in the prior art, the purpose of the present application is to provide a use of fumaric acid esters and pharmaceutically acceptable salts thereof in preparing a pharmaceutical for treating a ferroptosis related diseases.

In order to achieve the above purpose, the present application adopts the following technical solution.

The present application provides a use of fumaric acid esters and pharmaceutically acceptable salts thereof in preparing a pharmaceutical for treating a ferroptosis related disease.

Dimethyl fumarate and its main metabolic activity product in vivo monomethyl fumarate may inhibit cellular reactive oxygen species (ROS) accumulation by activating a KEAP1-NRF2-ARE signal pathway which plays an important role in a cellular antioxidant mechanism, thereby withstanding the cell peroxidative damage. However, the prior art has no disclosure of use of dimethyl fumarate and monomethyl fumarate in treatment of ferroptosis related diseases, while they have a broad application prospect as a new-type pharmaceutical for treating ferroptosis-related diseases by using the ferroptosis as a target.

In the present application, the fumaric acid esters includes dimethyl fumarate and/or monomethyl fumarate.

The dosage form of the pharmaceutical includes tablets, powders, granules, capsules, injections, sprays, films, suppositories, nose drops or pills.

The administration route of the pharmaceutical comprises intravenous injection, intraperitoneal injection, intramuscular injection, subcutaneous injection, oral administration, sublingual administration, and nasal administration or transdermal administration.

In the present application, the ferroptosis related disease includes liver disease, neurological disease, tumor, acute renal failure, ischemia-reperfusion injury, or iron metabolism-related disease.

In the present application, the liver disease is a liver disease caused by an abnormal level of a ferroptosis related factor.

Preferably, the liver disease includes hemochromatosis, primary biliary cholangitis, and liver fibrosis.

In the present application, the neurological disease includes Parkinson's disease, Alzheimer's disease, periventricular leukomalacia, Huntington's disease, motor neurodegeneration, stroke or traumatic brain injury.

In the present application, the tumor includes head and neck malignant tumors, breast cancer, esophageal cancer, liver cancer, pancreatic cancer, kidney cancer or diffuse large B-cell lymphoma.

In the present application, the ischemia-reperfusion injury includes myocardial ischemia-reperfusion injury, liver ischemia-reperfusion injury, or renal ischemia-reperfusion injury.

In the present application, the iron metabolism related disease includes atherosclerosis or diabetes.

In the present application, the fumaric acid esters and pharmaceutically acceptable salts thereof are loaded on a pharmaceutical carrier.

The fumaric acid esters and pharmaceutically acceptable salts thereof may be loaded on a common pharmaceutical carrier as a pharmaceutical for the treatment of the ferroptosis related disease, to achieve better biocompatibility, biological safety and efficacy. The common pharmaceutical carrier includes liposomes, micelles, dendrimers, microspheres, microcapsules, etc.

In the present application, the fumaric acid esters and pharmaceutically acceptable salts thereof are contained in a pharmaceutical composition.

The fumaric acid esters and pharmaceutically acceptable salts thereof may also be used in combination with other pharmaceuticals or pharmaceutical excipients to achieve the treatment of the ferroptosis related disease.

Compared with the prior art, the present application at least has the following beneficial effects.

The present application provides a new use of fumaric acid esters and pharmaceutically acceptable salts thereof in preparing pharmaceuticals for treatment of ferroptosis related diseases, a theoretical basis for researching the treatment strategies of the ferroptosis diseases, and a new outlet for preparing pharmaceuticals for treating ferroptosis related diseases.

### BRIEF DESCRIPTION OF THE DRAWINGS

FIG. 1 is an effect result diagram of dimethyl fumarate on GSH level and GSSH level of a HepG2 cell.

FIG. 2 is an effect result diagram of monomethyl fumarate on GSH level and GSSH level of a HepG2 cell.

FIG. 3 is an effect result diagram of dimethyl fumarate and monomethyl fumarate on a cell membrane lipid peroxidation level caused by ethanol.

FIG. 4 is an effect result diagram of dimethyl fumarate and monomethyl fumarate on up-regulation of a cell membrane lipid peroxidation level caused by DOX.

FIG. 5 is an effect result diagram of dimethyl fumarate and monomethyl fumarate on down-regulation of a cell GPX4 protein level caused by ethanol.

FIG. 6 is an effect result diagram of dimethyl fumarate and monomethyl fumarate on down-regulation of a cell GPX4 protein level caused by DOX.

FIG. 7 is a pathological result diagram of mouse liver tissue H&E staining, and Fig. a, b, c and d are a control group, a model group, a model+low-dose dimethyl fumarate group and a model+high-dose dimethyl fumarate group respectively.

FIG. 8 is a pathological result diagram of mouse liver tissue anti-GPX4 immunohistochemistry, and Fig. a, b, c, and d are a control group, a model group, a model+low-dose dimethyl fumarate group and a model+high-dose dimethyl fumarate group respectively.

FIG. 9 is a pathological result diagram of mouse liver tissue anti-4-HNE immunohistochemistry, and Fig. a, b, c, and d are a control group, a model group, a model+low-dose dimethyl fumarate group and a model+high-dose dimethyl fumarate group respectively.

FIG. 10 is a GPX4 protein level result diagram of mouse liver tissue Western Blot detection, and Fig. a, b, c, and d are a control group, a model group, a model+low-dose dimethyl fumarate group and a model+high-dose dimethyl fumarate group respectively.

FIG. 11 is a pathological result diagram of mouse liver tissue H&E staining, and Fig. a, b, c, and d are a control group, a model group, a model+low-dose dimethyl fumarate group and a model+high-dose dimethyl fumarate group respectively.

FIG. 12 is a pathological result diagram of mouse liver tissue anti-GPX4 immunohistochemistry, and Fig. a, b, c, and d are a control group, a model group, a model+low-dose dimethyl fumarate group and a model+high-dose dimethyl fumarate group respectively.

FIG. 13 is a pathological result diagram of mouse liver tissue anti-4-HNE immunohistochemistry, and Fig. a, b, c, and d are a control group, a model group, a model+low-dose dimethyl fumarate group and a model+high-dose dimethyl fumarate group respectively.

FIG. 14 is a pathological result diagram of mouse liver tissue Sirius Red staining, and Fig. a, b, c, and d are a control group, a model group, a model+low-dose dimethyl fumarate group and a model+high-dose dimethyl fumarate group respectively.

### DETAILED DESCRIPTION OF THE EMBODIMENTS

Technical schemes of the present application are further described below through specific examples. It should be understood by those skilled in the art that the described examples are merely to help understand the present application, and should not be regarded as specific limitation to the present application.

Materials used in the following tests include:

Dimethyl fumarate (Sigma-Aldric, 242926; 10 μM), monomethyl fumarate (Sigma-Aldrich, 651419; 10 μM), ethanol (Sigma-Aldrich, E7023; 80 mM), doxorubicin (Solarbio, D8740; 10 μM), ferroptosis inhibitor Ferrostatin-1 (Sigma-Aldric, SML0583, 1 μM), ferroptosis activator Erastin (Selleck, S7242; 10 μM), NRF2 antibody (Proteintech, 16396-1-AP; 1:1000), GXP4 antibody (Abcam, ab125066; 1:1000), ACTB/6-actin antibody (Proteintech, 20536-1-AP; 1:1000), GXP4 antibody (Abcam, ab125066; 1:100), 4-HNE antibody (Abcam, ab46545; 1:100).

Human hepatocytes HepG2 and LO2 cells are obtained from the cell bank of Xiangya Hospital. The medium is DMEM medium (HyClone, SH30022.01) and RPMI medium (HyClone, SH30809.01) containing 10% fetal bovine serum (Gibco, 10091148) and 100 U/mL ampicillin+streptomycin double-antibody (Gibco, 10378016). The cell incubator conditions are 37° C., constant temperature and humidity, and constant carbon dioxide concentration of 5%.

### EXAMPLE 1

### In Vitro Inhibition Test of Hepatocyte Ferroptosis

### (1) GSH and GSSG Level Determination Test

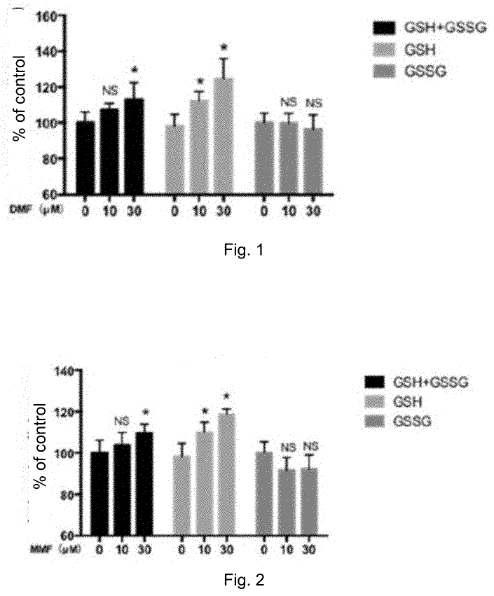

Monolayer adherent HepG2 cells are digested with a trypsin into single-cell suspension, and the cells are plated on a 96-well plate at 4000 cells/well. After the cells are adhered for 4-6 h, DMSO, 10 μM of dimethyl fumarate, 30 μM of dimethyl fumarate, 10 μM of monomethyl fumarate and 30 μM of monomethyl fumarate are respectively added to the cell culture plate, and incubated for 20 h, GSH/GSSG-Glo™ Assay Kit (Promega, V6611) is used to detect the total GSH and GSSH of the cells. Specific operations are performed according to the instructions, and the experiment is repeated for three times. Results are shown in FIG. 1 and FIG. 2.

It may be seen from the results in the figure that the dimethyl fumarate and monomethyl fumarate can significantly up-regulate the GSH level and GSSH level of the HepG2 cells in a concentration-dependent manner. Along with the increase of the fumarate concentration, their up-regulation levels are also increased (the dimethyl fumarate and monomethyl fumarate in the drawings of the description are respectively represented by DMF and MMF).

### (2) Determination Test of Cell Membrane Lipid Peroxidation Level

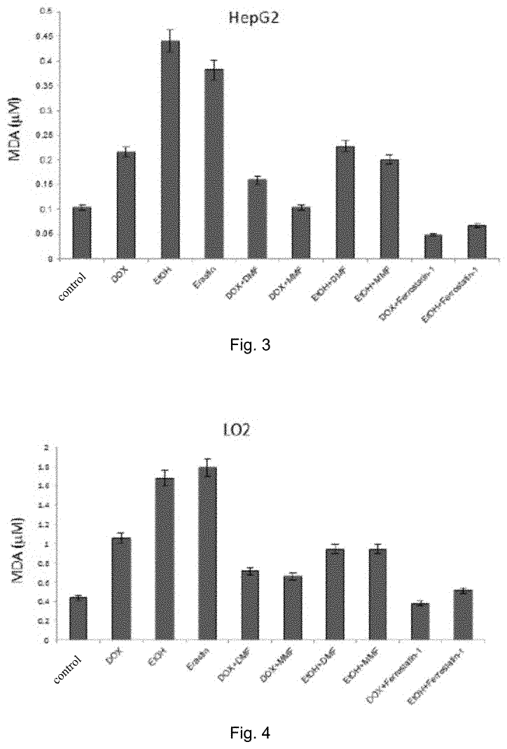

DMSO, 1 μM of Erastin, 80 mM of ethanol, 10 μM of DOX, 10 μM DOX+10 μM dimethyl fumarate, 10 μM DOX+10 μM monomethyl fumarate, 10 μM DOX+1 μM Ferrostatin-1, 80 mM ethanol+10 μM dimethyl fumarate, 80 mM ethanol+10 μM monomethyl fumarate, and 80 mM ethanol+1 μM Ferrostatin-1 are respectively added to monolayer adherent hepatocytes HepG2 and LO2 cells. After being incubated for 6 h, the cells are gently rinsed with PBS buffer pre-cooled at 4° C. for 3 times, and every 1 million cells are homogenized with 0.1 mL of IP lysate (Beyotime, P0013), and supernatant is collected after centrifugation at 4° C. The cell membrane lipid peroxidation level is measured by MDA detection kit (Beyotime, S0131), specific operations are performed according to the instructions, and the test is repeated for three times. Results are shown in FIG. 3 and FIG. 4.

It may be seen from the results in the figure that the stimulation of ethanol and DOX can significantly up-regulate the cell membrane lipid peroxidation levels of HepG2 and LO2, indicating that ethanol and DOX can cause occurrence of cell ferroptosis. The dimethyl fumarate and monomethyl fumarate can significantly inhibit the cell membrane lipid peroxidation levels caused by the ethanol and DOX, it is indicated that the dimethyl fumarate and monomethyl fumarate may inhibit the occurrence of the cell ferroptosis in vitro (the dimethyl fumarate and monomethyl fumarate in the drawings of the description are respectively represented by DMF and MMF).

### (3) Western Blot Test 1

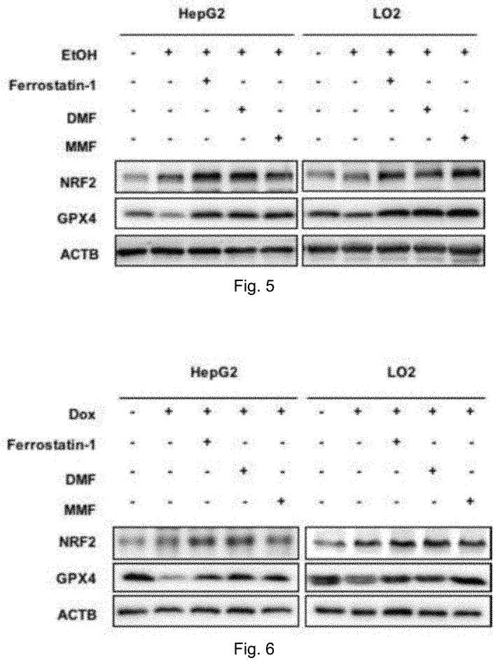

80 mM of ethanol, 80 mM ethanol+10 μM dimethyl fumarate, 80 mM ethanol+10 μM monomethyl fumarate, and 80 mM ethanol+1 μM Ferrostatin-1 are respectively added to monolayer adherent hepatocytes HepG2 and LO2 cells. After being incubated for 6 h, the cells are gently rinsed with PBS buffer pre-cooled at 4° C. for 3 times, and then extracted for total protein of the cells with RI PA lysate (Beyotime, P0013B). Specific operations are performed according to the instructions. After the concentration of the extracted protein is determined by BCA protein concentration determination kit (Beyotime, P0010S), it is stored at −20° C. for later use. Each group of protein sample and 2×Loading Buffer is prepared at a volume ratio of 1:1, and placed in a metal bath at 100° C. to boil and denature for 10 min, then placed on ice to cool for 5 min, and a sample is waited to be loaded. Specific operations are performed according to the instructions. The test is repeated for three times. Results are shown in FIG. 5.

It may be seen from the results in the figure that the stimulation of ethanol can significantly down-regulate the GPX4 protein levels of HepG2 and LO2 cells, it is indicated that the ethanol may cause the occurrence of the cell ferroptosis. The dimethyl fumarate and monomethyl fumarate may significantly up-regulate the GPX4 protein level of the cells, it is indicated that the dimethyl fumarate and monomethyl fumarate may inhibit the occurrence of the cell ferroptosis in vitro (the dimethyl fumarate and monomethyl fumarate in the drawings of the description are respectively represented by DMF and MMF).

### (4) Western Blot Test 2

10 μM of DOX, 10 μM DOX+10 μM dimethyl fumarate, 10 μM DOX+10 μM monomethyl fumarate, and 10 μM DOX+1 μM Ferrostatin-1 are respectively added to monolayer adherent hepatocytes HepG2 and LO2 cells. After being incubated for 6 h, the cells are gently rinsed with PBS buffer pre-cooled at 4° C. for 3 times, and then a total protein of the cells is extracted with RIPA lysate (Beyotime, P0013B). Specific operations are performed according to the instructions. After the concentration of the extracted protein is determined by BCA protein concentration determination kit (Beyotime, P0010S), it is stored at −20° C. for later use. Each histone sample and 2×Loading Buffer are prepared at a volume ratio of 1:1, and placed in a metal bath at 100° C. to boil and denature for 10 min, then placed on ice to cool for 5 min, and a sample is waited to be loaded. Specific operations are performed according to the instructions. The test is repeated for three times. Results are shown in FIG. 6.

It may be seen from the results in the figure that stimulation of DOX may significantly down-regulate the GPX4 protein levels of HepG2 and LO2 cells, indicating that DOX may cause the occurrence of the cell ferroptosis. The dimethyl fumarate and monomethyl fumarate may significantly up-regulate the GPX4 protein level of the cells, it is indicated that the dimethyl fumarate and monomethyl fumarate may inhibit the occurrence of the cell ferroptosis in vitro (the dimethyl fumarate and monomethyl fumarate in the drawings of the description are respectively represented by DMF and MMF).

### EXAMPLE 2

### Inhibition Test of Ferroptosis in Mouse Liver of Alcoholic Fatty Liver Disease Model

24 8-week-old SPF-grade C57BL/6 male mice are randomly divided into 4 groups, namely a blank group, a model group, a model+low-dose dimethyl fumarate (100 mg/Kg) group and a model+high-dose dimethyl fumarate (200 mg/Kg) group. The mice are let free to drink Lieber-DeCarli control diet for 5 days to gradually adapt to liquid diet and tube feeding. Subsequently, the model group is allowed to drink freely with 5% (volume/volume) standard Lieber-DeCarli alcohol liquid diet for 10 days, while the control group uses isocaloric control diet. From the 11-th day, the model+administration group is respectively administrated with dimethyl fumarate (100 mg/Kg) and dimethyl fumarate (200 mg/Kg) by intragastric administration once a day for 10 consecutive days. After that, the mice are anesthetized with 5% chloral hydrate in abdominal cavity, and liver tissues are collected, partly fixed in 10% formalin, and partly stored in liquid nitrogen. Then the following tests are separately performed (the dimethyl fumarate in the drawings of the description is represented by DMF):

### (1) Observation of Pathological Results of Mouse Liver Tissue H&E Staining

A specific method is as follows: a mouse liver tissue sample fixed in 10% formalin is dehydrated, paraffin-immersed and embedded, and the paraffin-embedded tissue is sectioned. Then, the tissue sections are baked in a constant temperature oven at 65° C. for 60 min to dissolve the paraffin, and then they are successively put in xylene to dewax and be transparent, and hydrated in ethanol solution with the concentrations from high to low. After the tissue sections are rinsed with PBS solution, they are stained with hematoxylin for 10 min, and differentiated with 1% hydrochloric acid alcohol. After running water returns to blue, they are stained with eosin for 3 min, and finally put in ethanol solution with the concentrations from low to high for dehydration, they are transparent in xylene, and the sections are blocked with neutral gum. After being dried at a room temperature, images are collected under a microscope and data analysis is performed.

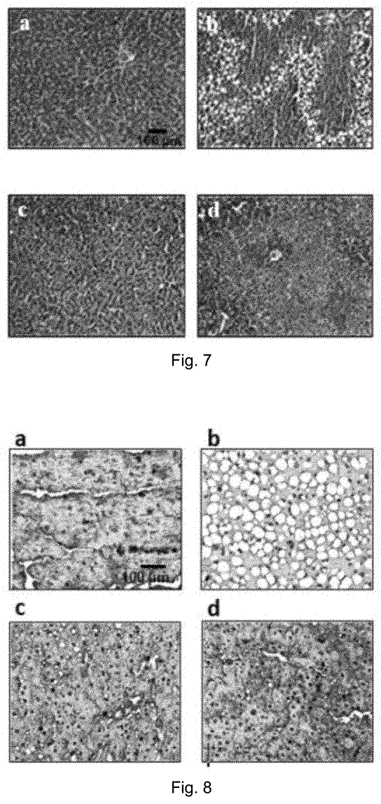

Results are shown in FIG. 7, Fig. a is a normal control group, Fig. b is an alcoholic liver disease model group, Fig. c is an alcoholic liver disease DMF (100 mg/Kg) treatment group, and Fig. d is an alcoholic liver disease DMF (200 mg/Kg) treatment group.

### (2) Anti-GPX4 Immunohistochemical Pathological Detection Test of Mouse Liver Tissue

A specific method is as follows: the paraffin-embedded tissue sections are baked in a constant temperature oven at 65° C. for 60 min to dissolve the paraffin, and then sequentially put in xylene to dewax and be transparent, and hydrated in ethanol solution with the concentrations from high to low. After the tissue sections are rinsed with PBS solution, a reagent A in a ready-to-use immunohistochemical hypersensitive UltraSensitive™ SP kit (Maixin Biotech Company, KIT-9707) is respectively added dropwise to remove an endogenous peroxidase from the tissues, and a reagent B is blocked by goat serum. The appropriate concentration of anti-GPX4 primary antibody solution is dropped on the tissue sections at 4° C. overnight. On the second day, the sections are taken out and placed at a room temperature for 30 min, then a reagent C (secondary antibody solution, it is noted that the source is the same as the primary antibody) and a reagent D are successively added dropwise, and specific operations are performed according to the instructions. Then DAB color development is performed, it is stained with hematoxylin, and differentiated with 1% hydrochloric acid alcohol. After running water returns to blue, it is dehydrated in ethanol solution with the concentrations from low to high, it is transparent in xylene, and the slide is blocked with neutral gum. After it is dried at a room temperature, images are collected under a microscope and data analysis is performed.

Results are shown in FIG. 8. Fig. a is a normal control group, Fig. b is an alcoholic liver disease model control group, Fig. c is an alcoholic liver disease DMF (100 mg/Kg) treatment group, and Fig. d is an alcoholic liver disease DMF (200 mg/Kg) treatment group.

### (3) Anti-4-HNE Immunohistochemical Pathological Detection Test of Mouse Liver Tissue

A specific method is as follows: the paraffin-embedded tissue sections are baked in a constant temperature oven at 65° C. for 60 min to dissolve the paraffin, and then sequentially put in xylene to dewax and be transparent, and hydrated in ethanol solution with the concentrations from high to low. After the tissue sections are rinsed with PBS solution, a reagent A in a ready-to-use immunohistochemical hypersensitive UltraSensitive™ SP kit (Maixin Biotech Company, KIT-9707) is respectively added dropwise to remove an endogenous peroxidase from the tissues, and a reagent B is blocked by goat serum. The appropriate concentration of anti-GPX4 primary antibody solution is dropped on the tissue sections at 4° C. overnight. On the second day, the sections are taken out and placed at a room temperature for 30 min, then a reagent C (secondary antibody solution, it is noted that the source is the same as the primary antibody) and a reagent D are successively added dropwise, and specific operations are performed according to the instructions. Then DAB color development is performed, it is stained with hematoxylin, and differentiated with 1% hydrochloric acid alcohol. After running water returns to blue, it is dehydrated in ethanol solution with the concentrations from low to high, it is transparent in xylene, and the slide is blocked with neutral gum. After it is dried at a room temperature, images are collected under a microscope and data analysis is performed.

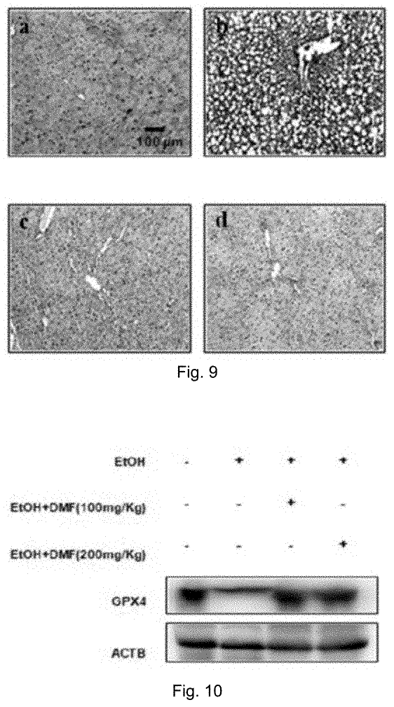

Results are shown in FIG. 9, Fig. a is a normal control group, Fig. b is an alcoholic liver disease model control group, Fig. c is an alcoholic liver disease DMF (100 mg/Kg) treatment group, and Fig. d is an alcoholic liver disease DMF (200mg/Kg)) treatment group.

### (4) GPX4 Protein Level Test of Western Blot Detection in Mouse Liver Tissue

A specific method is as follows: a mouse liver sample stored in liquid nitrogen is taken out, and proteins are extracted with 0.5% NP-40 lysate, and stored at −20° C. for later use. Each group of protein sample and 2×Loading Buffer is prepared at a volume ratio of 1:1, placed in a metal bath at 100° C. to boil and denature for 10 min, and then placed on ice to cool for 5 min, and a sample is loaded. 15% SDS-PAGE gel electrophoresis, transfer membrane, primary antibody and secondary antibody immunoreaction, chemiluminescence development and gel image analysis are performed in sequence. Specific operations are performed according to the instructions, and the test is repeated for three times.

Results are shown in FIG. 10.

It may be seen from the results in FIG. 7-10 that: there are a large number of lipid droplets in the liver tissues of the alcoholic liver disease model group, fatty degeneration of hepatocytes is moderate, and accompanied with a small number of ballooning degeneration and punctate necrosis of the hepatocytes, and pericentral periphlebitis, the GPX4 protein level is significantly down-regulated, and the 4-HNE protein level is significantly up-regulated, it hints the occurrence of the ferroptosis in vivo. After the dimethyl fumarate (100 mg/Kg or 200 mg/Kg) is administrated to the model mice once a day by introgastric administration for 10 consecutive days, it is observed that the fatty degeneration of the hepatocytes and the pericentral periphlebitis are significantly reduced, and even tended to the liver tissue of the normal control group, and the GPX4 protein level is significantly up-regulated, the 4-HNE protein level is significantly down-regulated, it is indicated that the dimethyl fumarate may significantly inhibit the ferroptosis in vivo.

### EXAMPLE 3

### Inhibition Test of Ferroptosis in Mouse Liver of Hepatic Fibrosis Model

36 7-week-old SPF-grade SD mice of 200-250 g are randomly divided into 4 groups, namely a control group, a model group, a model+low-dose dimethyl fumarate (15 mg/Kg) group and a model+high-dose dimethyl fumarate (25 mg/Kg) group. In the model group, subcutaneous injection of 0.3 ml/100 g of 40% CCI4 peanut oil solution is used for modeling twice a week. It is started from the second week, 10% alcohol is used as the only beverage, the mice are fed with a corn meal feed mixed with 0.5% cholesterol, and modeling is continuously performed for 8 weeks. At the same time of modeling, the model+administration group are respectively given with dimethyl fumarate (15 mg/Kg) and dimethyl fumarate (25 mg/Kg) once a day by intragastric administration for 8 consecutive weeks. After that, the mice are anesthetized with 5% chloral hydrate in abdominal cavity, and the liver tissues are collected, partly fixed in formalin, and partly stored in liquid nitrogen. Then the following tests are separately performed (the dimethyl fumarate in the drawings of the description is represented by DMF):

### (1) Observation of Pathological Results of Mouse Liver Tissue H&E Staining

A specific method is as follows: a mouse liver tissue sample fixed in 10% formalin is dehydrated, paraffin-immersed and embedded, and the paraffin-embedded tissue is sectioned. Then, the tissue sections are baked in a constant temperature oven at 65° C. for 60 min to dissolve the paraffin, and then they are successively put in xylene to dewax and be transparent, and hydrated in ethanol solution with the concentrations from high to low. After the tissue sections are rinsed with PBS solution, they are stained with hematoxylin for 10 min, and differentiated with 1% hydrochloric acid alcohol. After running water returns to blue, they are stained with eosin for 3 min, and finally put in ethanol solution with the concentrations from low to high for dehydration, they are transparent in xylene, and the sections are blocked with neutral gum. After being dried at a room temperature, images are collected under a microscope and data analysis is performed.

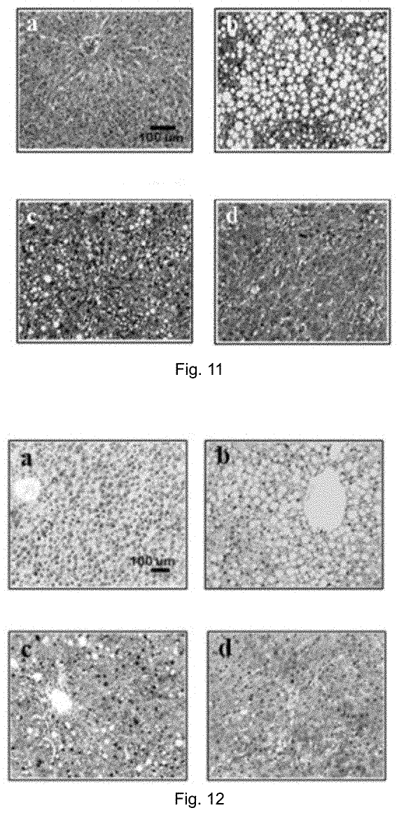

Results are shown in FIG. 11, Fig. a is a normal control group, Fig. b is a liver fibrosis model control group, Fig. c is a liver fibrosis DMF (15 mg/Kg) treatment group, and Fig. d is a liver fibrosis DMF (25 mg/Kg) treatment group.

### (2) Anti-GPX4 Immunohistochemical Pathological Detection Test of Mouse Liver Tissue

A specific method is as follows: the paraffin-embedded tissue sections are baked in a constant temperature oven at 65° C. for 60 min to dissolve the paraffin, and then sequentially put in xylene to dewax and be transparent, and hydrated in ethanol solution with the concentrations from high to low. After the tissue sections are rinsed with PBS solution, a reagent A in a ready-to-use immunohistochemical hypersensitive UltraSensitive™ SP kit (Maixin Biotech Company, KIT-9707) is respectively added dropwise to remove an endogenous peroxidase from the tissues, and a reagent B is blocked by goat serum. The appropriate concentration of anti-GPX4 primary antibody solution is dropped on the tissue sections at 4° C. overnight. On the second day, the sections are taken out and placed at a room temperature for 30 min, then a reagent C (secondary antibody solution, it is noted that the source is the same as the primary antibody) and a reagent D are successively added dropwise, and specific operations are performed according to the instructions. Then DAB color development is performed, it is stained with hematoxylin, and differentiated with 1% hydrochloric acid alcohol. After running water returns to blue, it is dehydrated in ethanol solution with the concentrations from low to high, it is transparent in xylene, and the slide is blocked with neutral gum. After it is dried at a room temperature, images are collected under a microscope and data analysis is performed.

Results are shown in FIG. 12, Fig. a is a normal control group, Fig. b is a liver fibrosis model control group, Fig. c is a liver fibrosis DMF (15 mg/Kg) treatment group, and Fig. d is a liver fibrosis DMF (25 mg/Kg) treatment group.

### (3) Anti-4-HNE Immunohistochemical Pathological Detection Test of Mouse Liver Tissue

A specific method is as follows: the paraffin-embedded tissue sections are baked in a constant temperature oven at 65° C. for 60 min to dissolve the paraffin, and then sequentially put in xylene to dewax and be transparent, and hydrated in ethanol solution with the concentrations from high to low. After the tissue sections are rinsed with PBS solution, a reagent A in a ready-to-use immunohistochemical hypersensitive UltraSensitive™ SP kit (Maixin Biotech Company, KIT-9707) is respectively added dropwise to remove an endogenous peroxidase from the tissues, and a reagent B is blocked by goat serum. The appropriate concentration of anti-GPX4 primary antibody solution is dropped on the tissue sections at 4° C. overnight. On the second day, the sections are taken out and placed at a room temperature for 30 min, then a reagent C (secondary antibody solution, it is noted that the source is the same as the primary antibody) and a reagent D are successively added dropwise, and specific operations are performed according to the instructions. Then DAB color development is performed, it is stained with hematoxylin, and differentiated with 1% hydrochloric acid alcohol. After running water returns to blue, it is dehydrated in ethanol solution with the concentrations from low to high, it is transparent in xylene, and the slide is blocked with neutral gum. After it is dried at a room temperature, images are collected under a microscope and data analysis is performed.

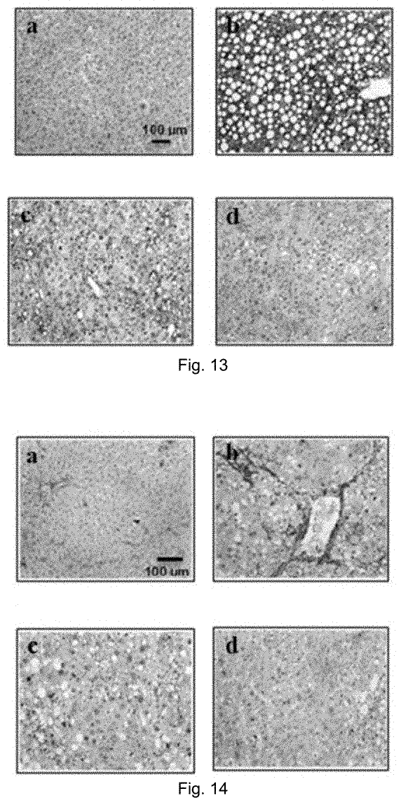

Results are shown in FIG. 13, Fig. a is a normal control group, Fig. b is a liver fibrosis model control group, Fig. c is a liver fibrosis DMF (15 mg/Kg) treatment group, and Fig. d is a liver fibrosis DMF (25 mg/Kg) treatment group.

### (4) Observation of Pathological Results of Sirius Red Staining in Mouse Liver Tissue

A specific method is as follows: a Sirius Red staining kit (Solarbio, G1470) is used, a mouse liver tissue is taken and fixed in 10% formalin stationary liquid for 8-12 h, it is conventionally dehydrated, embedded, and sectioned, the thickness is about 6 μm, and it is conventionally dewaxed to water. It is stained with Weigert iron hematoxylin staining solution for 10-20 min, and differentiated for a few seconds with hydrochloric acid alcohol. Running water returns to blue for 15 min, and it is washed once with ddH2O. It is drop-stained with Sirius Red staining solution for 1 h, slightly rinsed with the running water, and conventionally dehydrated to be transparent, and the slide is blocked with neutral gum for observation.

Results are shown in FIG. 14, Fig. a is a normal control group, Fig. b is a liver fibrosis model control group, Fig. c is a liver fibrosis DMF (15 mg/Kg) treatment group, and Fig. d is a liver fibrosis DMF (25 mg/Kg) treatment group.

It may be seen from the results in FIG. 11-14 that: there are a large number of lipid droplets in the mouse liver tissue of the fibrosis model group, fatty degeneration of hepatocytes is moderate, and accompanied with a few ballooning degeneration and punctate necrosis of the hepatocytes, periportal inflammation, fibrosis expansion, localized perisinusitis and intralobular fibrosis, it hints that the liver has moderate fibrosis, the GPX4 protein level is significantly down-regulated, and the 4-HNE protein level is significantly up-regulated, it hints that the ferroptosis occurs in the mouse liver tissue of the fibrosis model group. A group of mice are treated with dimethyl fumarate (15 mg/Kg or 25 mg/Kg) once a day. After 8 weeks of continuous administration, it is observed that the above pathological changes are significantly reduced, and even tended to the liver tissue of the normal control group. In addition, the GPX4 protein level is significantly up-regulated, and the 4-HNE protein level is significantly down-regulated, it is indicated that the dimethyl fumarate may significantly inhibit the occurrence of the ferroptosis in vivo.

It is declared by the applicant that the present application uses the above examples to describe the use of fumaric acid esters and pharmaceutically acceptable salts thereof in preparing drugs for treating the ferroptosis related disease, but the present application is not limited to the above examples, namely it does not mean that the present application must rely on the above examples to be implemented. It should be understood by those skilled in the art that any improvements to the present application, equivalent replacements to each raw material of a product of the present application, addition of auxiliary components, selection of specific modes, etc. shall fall within scopes of protection and disclosure of the present application.

The preferred implementation modes of the present application are described in detail above, but the present application is not limited to specific details in the above implementation modes. Within a scope of a technical concept of the present application, a variety of simple modifications may be made to the technical schemes of the present application, and these simple modifications all belong to the protection scope of the present application.

In addition, it should be noted that various specific technical features described in the above specific implementation modes may be combined in any suitable forms in the case without conflicting. In order to avoid unnecessary repetition, various possible combinations are not described separately in the present application.

What is claimed is:

1. Use of fumaric acid esters or pharmaceutically acceptable salts in preparing a pharmaceutical for treating ferroptosis-related diseases.

2. The use as claimed in claim 1, wherein fumaric acid esters comprise dimethyl fumarate and/or monomethyl fumarate.

3. The use as claimed in claim 1, wherein the dosage form of the pharmaceutical comprises tablets, powders, granules, capsules, injections, sprays, films, suppositories, nose drops or pills.

4. The use as claimed in claim 1, wherein the administration route of the pharmaceutical comprises intravenous injection, intraperitoneal injection, intramuscular injection, subcutaneous injection, oral administration, sublingual administration, and nasal administration or transdermal administration.

5. The use as claimed in claim 1, wherein the ferroptosis-related diseases comprise liver diseases, neurological diseases, cancers, acute renal failure, ischemia-reperfusion injuries, or iron metabolism-related diseases.

6. The use as claimed in claim 5, wherein the liver disease is caused by abnormal level of a ferroptosis-related factor.

7. The use as claimed in claim 6, wherein the liver diseases comprise hemochromatosis, primary biliary cholangitis, and liver fibrosis.

8. The use as claimed in claim 5, wherein the neurological diseases comprise Parkinson's disease, Alzheimer's disease, periventricular leukomalacia, Huntington's disease, motor neurodegeneration, stroke or traumatic brain injury.

9. The use as claimed in claim 5, wherein the cancers comprise head and neck malignant tumors, breast cancer, esophageal cancer, liver cancer, pancreatic cancer, kidney cancer or diffuse large B-cell lymphoma.

10. The use as claimed in claim 5, wherein the ischemia-reperfusion injuries comprise myocardial ischemia-reperfusion injury, liver ischemia-reperfusion injury, or renal ischemia-reperfusion injury.

11. The use as claimed in claim 5, wherein the iron metabolism related disease comprises atherosclerosis or diabetes.

12. The use as claimed in claim 1, wherein fumaric acid esters and/or pharmaceutically acceptable salts are loaded on a pharmaceutical carrier.

13. The use as claimed in claim 1, wherein fumaric acid esters and/or pharmaceutically acceptable salts are contained in a pharmaceutical composition.

14. The use as claimed in claim 2, wherein the ferroptosis-related diseases compose liver diseases, neurological diseases, tumor acute renal failure, ischemia-reperfusion injuries, or iron metabolism-related diseases.

15. The use as claimed in claim 14, wherein the liver disease is caused by abnormal level of a ferroptosis related factor.

16. The use as claimed in claim 15, wherein the liver diseases comprise hemochromatosis, primary biliary cholangitis, and liver fibrosis.

17. The use as claimed in claim 14, wherein the neurological diseases comprise Parkinson's disease, Alzheimer's disease, periventricular leukomalacia, Huntington's disease, motor neurodegeneration, stroke or traumatic brain injury.

18. The use as claimed in claim 14, wherein the cancers comprise head and neck malignant tumors breast cancer, esophageal cancer, liver cancer, pancreatic cancer, kidney cancer or diffuse large B-cell lymphoma.

19. The use as claimed in claim 14, wherein the ischemia-reperfusion injuries comprise myocardial ischemia-reperfusion injury, liver ischemia-reperfusion injury, or renal reperfusion injury.

20. The use as claimed In claim 14, wherein the iron metabolism related diseases comprise atherosclerosis or diabetes.
